(19) United States
(12) Patent Application Publication
(10) Pub. No.: US 2017/0049320 A1
(43) Pub. Date: Feb. 23, 2017

(54) OPTICAL MEASURING APPARATUS AND METHOD OF OUTPUTTING LIGHT AND RECEIVING THE LIGHT

(71) Applicant: FUJI XEROX CO., LTD., Tokyo (JP)

(72) Inventors: Saori NISHIZAKI, Kanagawa (JP); Taku KINOSHITA, Kanagawa (JP); Kazutaka TAKEDA, Kanagawa (JP); Hideaki OZAWA, Kanagawa (JP); Kazuyuki MATSUSHITA, Kanagawa (JP); Kouhei YUKAWA, Kanagawa (JP); Sho KIMURA, Kanagawa (JP); Hideo NAKAYAMA, Kanagawa (JP)

(73) Assignee: FUJI XEROX CO., LTD., Tokyo (JP)

(21) Appl. No.: 15/200,039

(22) Filed: Jul. 1, 2016

(30) Foreign Application Priority Data
Aug. 18, 2015 (JP) 2015-160919

(51) Int. Cl. A61B 3/117; A61B 3/00

(52) U.S. Cl. CPC A61B 3/117; A61B 3/0083; A61B 3/0091; A61B 3/0041

(57) ABSTRACT

An optical measuring apparatus includes a light output device that outputs light so as to cross an anterior chamber of an eyeball of a subject, a light receiving device that receives the light having crossed the anterior chamber, and a positioning device that positions the light output device and the light receiving device at such positions that, when the eyeball is adducted, the light output from the light output device crosses the anterior chamber and is received by the light receiving device.

### CROSS-REFERENCE TO RELATED APPLICATIONS

This application is based on and claims priority under 35 USC 119 from Japanese Patent Application No. 2015-160919 filed Aug. 18, 2015.

### BACKGROUND

### Technical Field

The present invention relates to an optical measuring apparatus and a method of outputting light and receiving the light.

### SUMMARY

According to an aspect of the present invention, an optical measuring apparatus includes a light output device that outputs light so as to cross an anterior chamber of an eyeball of a subject, a light receiving device that receives the light having crossed the anterior chamber, and a positioning device that positions the light output device and the light receiving device at such positions that, when the eyeball is adducted, the light output from the light output device crosses the anterior chamber and is received by the light receiving device.

### BRIEF DESCRIPTION OF THE DRAWINGS

Exemplary embodiments of the present invention will be described in detail based on the following figures, wherein:

FIG. 1 illustrates an example of a structure of an optical measuring apparatus to which an exemplary embodiment is applied;

FIG. 2 illustrates a method of measuring a rotational angle of a polarization plane due to optically active substances contained in aqueous humor in an anterior chamber by using the optical measuring apparatus;

FIGS. 3A and 3B illustrate outline structures of an eyelid retainer and an inner canthus presser;

FIGS. 4A to 4D illustrate a detailed structure of the eyelid retainer;

FIGS. 5A and 5B illustrate a detailed structure of the inner canthus presser;

FIG. 6 illustrates operations of the eyelid retainer and the inner canthus presser;

FIGS. 7A and 7B illustrate disposition of the optical measuring apparatus;

FIGS. 8A and 8B illustrate structures of optical measuring apparatuses according to other exemplary embodiments;

FIG. 9 illustrates a structure of an optical measuring apparatus according to yet another exemplary embodiment.

### DETAILED DESCRIPTION

Exemplary embodiments according to the present invention will be described below with reference to the accompanying drawings.

### First Exemplary Embodiment

### An Optical Measuring Apparatus 1

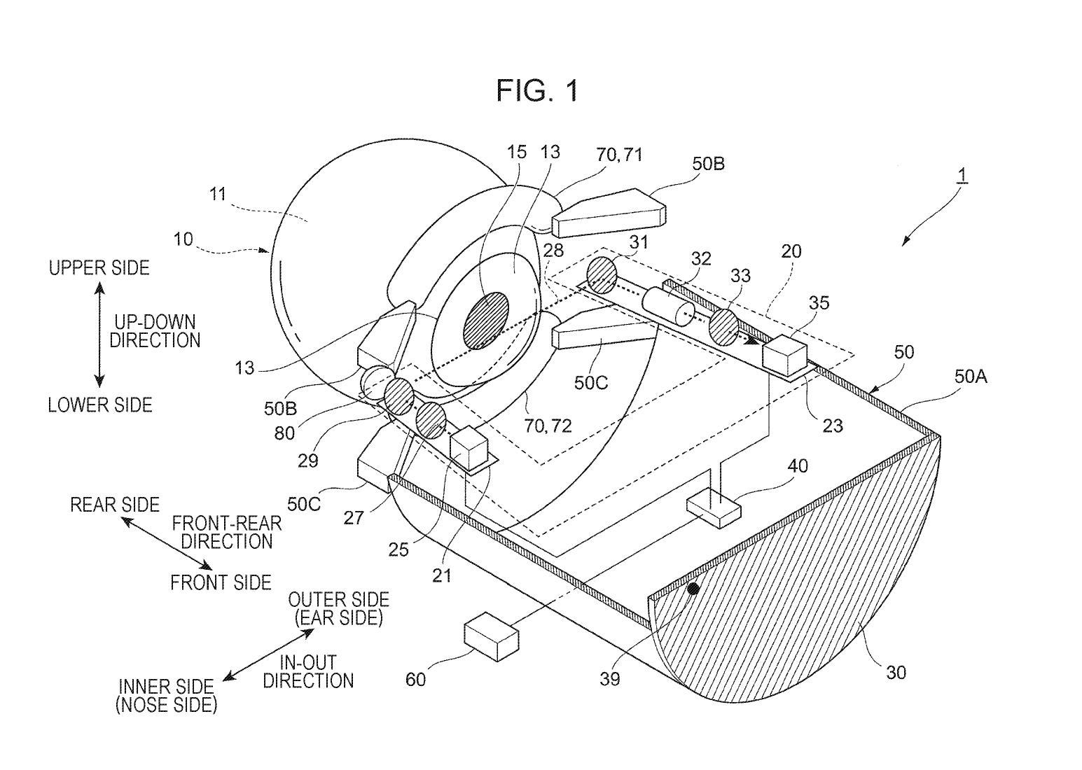

FIG. 1 illustrates an example of a structure of an optical measuring apparatus 1 to which a first exemplary embodiment is applied. The optical measuring apparatus 1 has a configuration that allows measurement to be performed by the optical measuring apparatus 1 held by the hand of a subject so as to attach (apply) the optical measuring apparatus 1 to his or her eyeball 10 (region around the eyeball 10). Here, the eyeball 10 of FIG. 1 is the left eye of the subject.

The optical measuring apparatus 1 includes an optical system 20, a display unit 30, a controller 40, a holding unit 50, a calculation unit 60, an eyelid retainer 70, and an inner canthus presser 80. The optical system 20 is used to measure characteristics of aqueous humor in an anterior chamber 13 of the eyeball 10 of the subject. The display unit 30 displays a guidance that guides the line of sight of the subject. The controller 40 controls the optical system 20 and the display unit 30. The holding unit 50 holds the optical system 20, the display unit 30, and the controller 40. The calculation unit 60 calculates the characteristics of the aqueous humor in accordance with data measured using the optical system 20. The eyelid retainer 70 is brought into contact with eyelids of the subject so as to retain the eyelids. The inner canthus presser 80 presses an inner canthus side of the eyelids of the subject.

It is noted that, in the following description, a direction extending between the upper side and the lower side of the optical measuring apparatus 1 illustrated in the page of FIG. 1 may be referred to as an “up-down direction”. Furthermore, a direction extending between the front side and the rear side of the subject illustrated in FIG. 1 may be referred to as a “front-rear direction”. Furthermore, a direction extending between the inner side (nose side, inner canthus side) and the outer side (ear side, outer canthus side) seen from the subject who uses the optical measuring apparatus 1 illustrated in FIG. 1 may be referred to as an “in-out direction”.

The characteristics of the aqueous humor measured by the optical measuring apparatus 1 to which the present exemplary embodiment is applied refer to, for example, a rotational angle (optical rotation α_{M}) of a polarization plane of linearly polarized light due to optically active substances contained in the aqueous humor and the degree of color absorbance (circular dichroism) with respect to circular polarized light. It is noted that the polarization plane of the linearly polarized light refers to a plane where an electric field oscillates in the linearly polarized light.

The optical system 20 includes a light emitting system 21 that emits light with which the anterior chamber 13 of the eyeball 10 is irradiated and a light receiving system 23 that receives the light having passed through the anterior chamber 13.

First, the light emitting system 21 includes a light emitter 25, a polarizer 27, and a first mirror 29.

The light emitter 25 may be a light source such as a light emitting diode (LED) or a lamp that has a large wavelength width or a light source such as a laser that has a small wavelength width. The light emitter 25 may output light at two or more wavelengths.

The polarizer 27 is, for example, a Nicol prism that allows passage of linearly polarized light of a predetermined polarization plane out of the light incident thereupon.

The first mirror 29 serving as an example of a light output device bends an optical path 28. The first mirror 29 may maintain the linearly polarized light as it is before and after reflection. The first mirror 29 is not necessarily provided when it is not required to bend the optical path 28.

Next, the light receiving system 23 includes a second mirror 31, a compensator 32, an analyzer 33, and a light receiver 35.

The second mirror 31 serving as an example of a light receiving device has a structure that is the same as or similar to that of the first mirror 29. The second mirror 31 bends the optical path 28. The second mirror 31 may maintain the linearly polarized light as it is before and after reflection. The second mirror 31 is not necessarily provided when it is not required to bend the optical path 28.

The compensator 32 is a magneto-optical element such as, for example, a Faraday element using a garnet and rotates the polarization plane of the linearly polarized light by using a magnetic field.

The analyzer 33 is an element that is the same as or similar to the polarizer 27. The analyzer 33 allows passage of linearly polarized light of a predetermined polarization plane.

The light receiver 35 is a light receiving element such as a silicon diode and outputs an output signal corresponding to the intensity of light.

The display unit 30 serving as an example of a display includes a display device that electronically displays an image. The display unit 30 displays a mark (target) 39 that is visible to the subject so as to guide a direction of the eyeball 10 (line of sight) to a predetermined direction. The display unit 30 displays the image of predetermined information such as the characteristics (density of the optically active substances and the like) of the aqueous humor calculated by the calculation unit 60.

The controller 40 controls the components of the optical system 20 such as the light emitter 25, the compensator 32, and the light receiver 35 so as to obtain measurement data relating to the characteristics of the aqueous humor. Furthermore, the controller 40 causes the display unit 30 to display the mark 39.

The holding unit 50 is a housing having a substantially cylindrical shape and holds the optical system 20 and the controller 40. The holding unit 50 has a configuration that allows the subject to hold the holding unit 50 by his or her own hand so as to attach the optical measuring apparatus 1 to his or her eyeball 10. For ease of viewing of the optical system 20, the cylinder of the holding unit 50 of FIG. 1 is cut along a plane parallel to the axial direction. The shape of the holding unit 50 may be another shape. For example, the holding unit 50 may have a barrel shape having a quadrangle or oblong section. Furthermore, a bottom surface on the opposite side to an attachment side of a barrel-shaped housing may be opened or may be closed by another component.

The calculation unit 60 receives the measurement data from the controller 40 and calculates the characteristics of the aqueous humor.

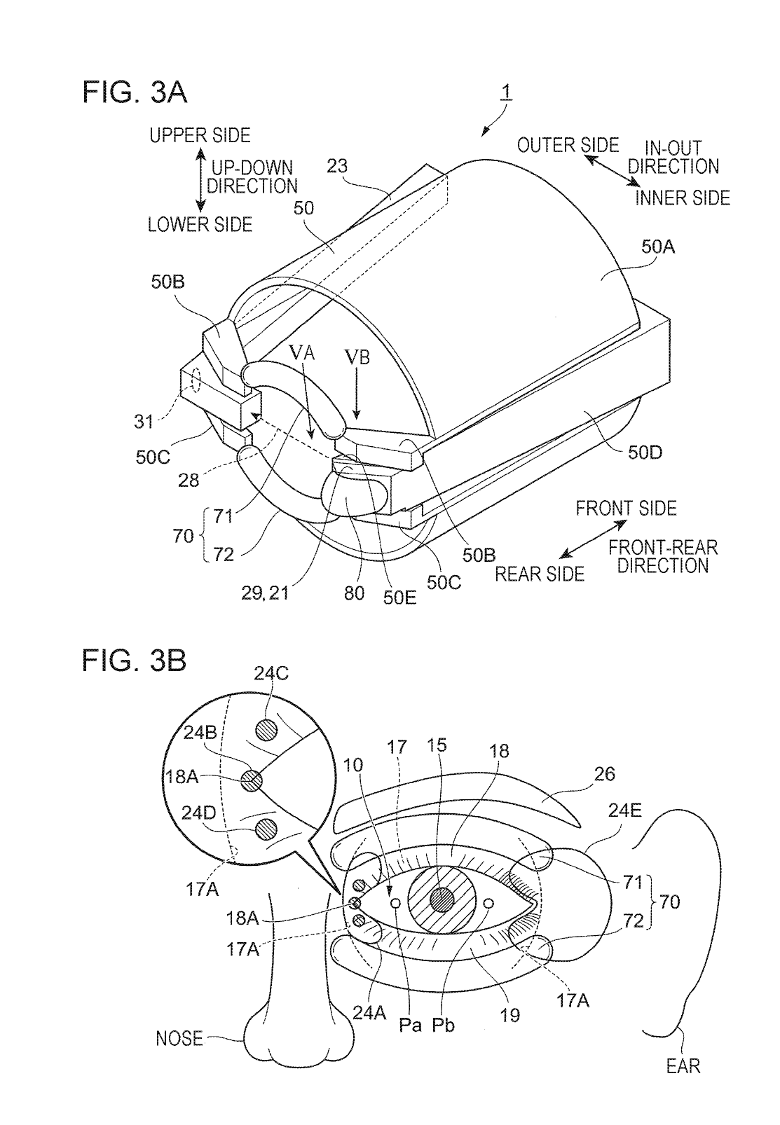

The eyelid retainer 70 serving as an example of a retaining device and an eyelid opening device is provided in the holding unit 50. The eyelid retainer 70 is brought into contact with eyelids (upper eyelid 18 and lower eyelid 19; see FIG. 3B that will be referred to later), thereby retaining the eyelids to keep the eyelids open. The structure of the eyelid retainer 70 will be described later.

The inner canthus presser 80 serving as an example of a presser is provided in the holding unit 50 and presses the eyelids toward the rear side. The structure of the inner canthus presser 80 will be described later.

### Measurement of the Aqueous Humor

Next, an example of calculation of glucose concentration in the aqueous humor by measuring the aqueous humor of the anterior chamber 13 using the optical measuring apparatus 1 is described.

The amount of insulin dosed to a diabetic patient is controlled in accordance with the glucose concentration in the blood. Thus, it is required to constantly grasp the glucose concentration in the blood of the diabetic patient. In order to measure the glucose concentration in the blood, a method may be adopted in which a slight amount of blood is collected by inserting an injection needle into, for example, an end of a finger. However, with this method, even when the amount of the collected blood is small, the patient may perceive pain. This may impose a mental burden on the patient. Accordingly, there is an increasing demand for non-invasive inspection methods that replace invasive inspection methods such as the insertion.

Here, the aqueous humor in the anterior chamber 13, which contains substantially the same components as serum, contains protein, glucose, ascorbic acid, and so forth. It is also known that there is a relationship between the glucose concentration in the blood and the glucose concentration in the aqueous humor. Furthermore, cell substances contained in the blood are generally not contained in the aqueous humor. This reduces the effect of light scattering. The protein, the glucose, the ascorbic acid, and so forth contained in the aqueous humor are optically active substances and have optical rotatory power.

Thus, with the optical measuring apparatus 1 to which the present exemplary embodiment is applied, the concentrations of the glucose and the like having optical rotatory power are optically measured by utilizing such aqueous humor.

### Setting of Optical Paths

In a method of optically measuring the concentrations or the like of the optically active substances such as glucose contained in the aqueous humor, two optical paths described below may be set.

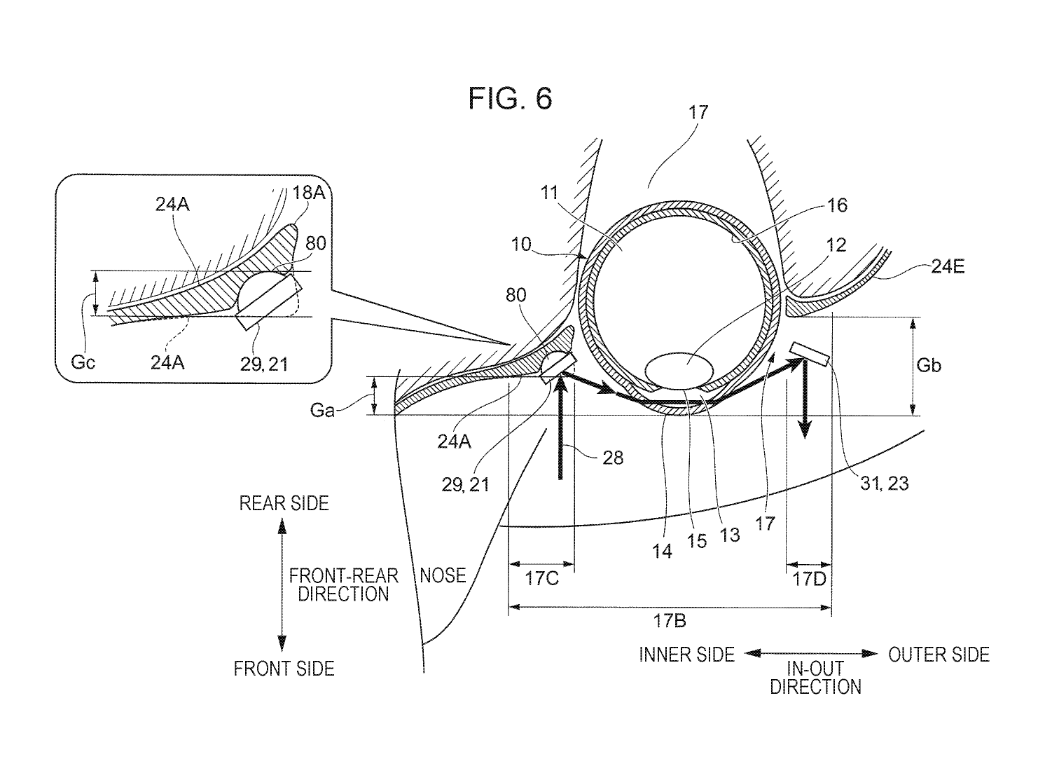

One of the optical paths is different from that of the structure of FIG. 1. In this optical path, light is caused to be nearly perpendicularly incident upon the eyeball 10, that is, in the front-rear direction, the light is reflected at an interface between a cornea 14 (see FIG. 6) and the aqueous humor or an interface between a crystalline lens 12 and the aqueous humor, and the reflected light is received (detected). The other optical path is, as of the structure illustrated in FIG. 1, the light is incident upon the eyeball 10 at an angle intersecting the front-rear direction, specifically, in a direction nearly parallel to the eyeball 10, and the light having passed through the aqueous humor in the anterior chamber 13 is received (detected).

As is the case with the former optical path in which the light is nearly perpendicularly incident upon the eyeball 10, the light may reach a retina 16 (see FIG. 6). In particular, when a laser, which has high coherency, is used for the light emitter 25, a situation in which the light reaches the retina 16 is not good.

In contrast, as is the case with the latter optical path, the optical path 28 through which the light is incident upon the eyeball 10 in the direction nearly parallel to the eyeball 10, the light is caused to path through so as to cross the anterior chamber 13 through the cornea 14, and the light having passed through the aqueous humor is received (detected). This may suppress the occurrences of a situation in which the light reaches the retina 16.

### Calculation of Concentrations of the Optically Active Substances

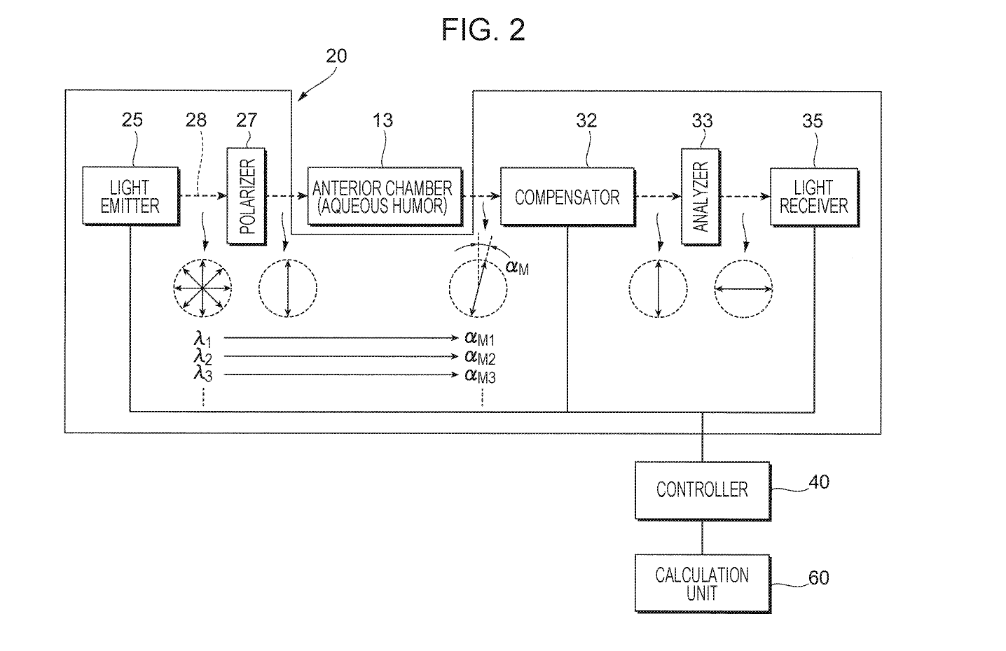

FIG. 2 illustrates a method of measuring the rotational angle (optical rotation) of the polarization plane due to the optically active substances contained in the aqueous humor in the anterior chamber 13 by using the optical measuring apparatus 1. Here, for ease of description, the optical path 28 is not bent (that is, a straight line), and accordingly, the first mirror 29 and the second mirror 31 are omitted from description.

Furthermore, in spaces between the light emitter 25, the polarizer 27, the anterior chamber 13, the compensator 32, the analyzer 33, and the light receiver 35 of FIG. 2, states of the polarized light seen in a light traveling direction are represented by arrows inside circles.

The light emitter 25 outputs light having random polarization planes. The polarizer 27 allows linearly polarized light of a predetermined polarization plane to pass therethrough. Referring to FIG. 2, as an example, linearly polarized light of a polarization plane parallel to the page of FIG. 2 passes.

The polarization plane of the linearly polarized light having passed through the polarizer 27 is rotated due to optically active substances contained in the aqueous humor in the anterior chamber 13. In FIG. 2, the polarization plane is rotated by an angle α_{M} (optical rotation α_{M}).

Next, the polarization plane having been rotated due to the optically active substances contained in the aqueous humor in the anterior chamber 13 is returned to an original state by applying a magnetic field to the compensator 32.

The linearly polarized light having passed through the analyzer 33 is received by the light receiver 35 and converted into the output signal corresponding to the intensity of the light.

Here, an example of the method of measuring the optical rotation α_{M} by using the optical system 20 is described.

Initially, in a state in which the light having been output from the light emitter 25 is not caused to pass through the anterior chamber 13, the compensator 32 and the analyzer 33 are set so as to minimize the output signal of the light receiver 35 by using the optical system 20 including the light emitter 25, the polarizer 27, the compensator 32, the analyzer 33, and the light receiver 35. In the example of FIG. 2, in the state in which the light is not caused to pass through the anterior chamber 13, the polarization plane of the linearly polarized light having passed through the polarizer 27 is perpendicular to the polarization plane passing through the analyzer 33.

Next, a state in which the light passes through the anterior chamber 13 is entered. As a result, the polarization plane is rotated due to the optically active substances contained in the aqueous humor in the anterior chamber 13. This changes the output signal from the light receiver 35 into a non-minimum value. Then, the magnetic field applied to the compensator 32 is set so as to minimize the output signal from the light receiver 35. That is, the polarization plane is rotated by the compensator 32 so as to be perpendicular to a polarization plane passing through the analyzer 33.

The angle by which the polarization plane has been rotated by the compensator 32 corresponds to the optical rotation α_{M} caused due to the optically active substances contained in the aqueous humor. Here, the relationship between the magnitude of the magnetic field applied to the compensator 32 and the angle of the polarization plane having been rotated is known in advance. Accordingly, the optical rotation α_{M} is found in accordance with the magnitude of the magnetic field applied to the compensator 32.

Specifically, beams of light of plural wavelengths (wavelengths λ₁, λ₂, λ₃ . . . ) are caused to be incident upon the aqueous humor in the anterior chamber 13 from the light emitter 25, and the optical rotations α_{M} (optical rotations α_{M1}, α_{M2}, α_{M3}, . . . ) corresponding to the respective wavelengths are obtained. Combinations of the wavelengths λ and the corresponding optical rotations α_{M} are input to the calculation unit 60, thereby the concentrations of the target optically active substances are calculated.

The concentrations of the optically active substances calculated by the calculation unit 60 may be displayed in the display unit 30 provided in the optical measuring apparatus 1 or output to another terminal device (not illustrated) such as a personal computer (PC) through an output device (not illustrated) provided in the optical measuring apparatus 1.

Additionally, the aqueous humor contains plural optically active substances as described above. Accordingly, the measured optical rotation α_{M} is the sum of the optical rotations α_{M} produced by the plural optically active substances. Thus, it is required to calculate the concentration of a target optically active substance from the measured optical rotation α_{M}. The concentration of the target optically active substance may be calculated by using a known method. Thus, description of the calculation of the concentration of the target optically active substance is omitted herein.

Furthermore, it is assumed in FIG. 2 that the polarization plane of the polarizer 27 and the polarization plane before passing through the analyzer 33 are parallel to the page of FIG. 2. However, when the polarization plane is rotated by the compensator 32 in advance, the polarization plane before passing through the analyzer 33 may be inclined relative to a plane parallel to the page of FIG. 2. That is, it is sufficient that, in a state in which the light does not pass through the aqueous humor in the anterior chamber 13, the compensator 32 and the analyzer 33 be set so as to minimize the output signal of the light receiver 35.

Furthermore, although the example using the compensator 32 is described as the method of obtaining the optical rotation α_{M} here, the optical rotation α_{M} may be obtained with a method other than the method using the compensator 32. Furthermore, although an orthogonally oriented polarizer method (although the compensator 32 is used), which is a most basic method of measuring the rotational angle (optical rotation α_{M}) of the polarization plane, is described, another measuring method such as a rotating analyzer method, a Faraday modulation method, or a retardation modulation method may be applied.

### Structures of the Eyelid Retainer 70 and the Inner Canthus Presser 80

FIGS. 3A and 3B illustrate outline structures of the eyelid retainer 70 and the inner canthus presser 80. More specifically, FIG. 3A is a perspective view of the optical measuring apparatus 1 seen from the rear side, and FIG. 3B illustrates the relationships between the positions of the eyelid retainer 70 and the inner canthus presser 80 and the position of the eyelids of the subject.

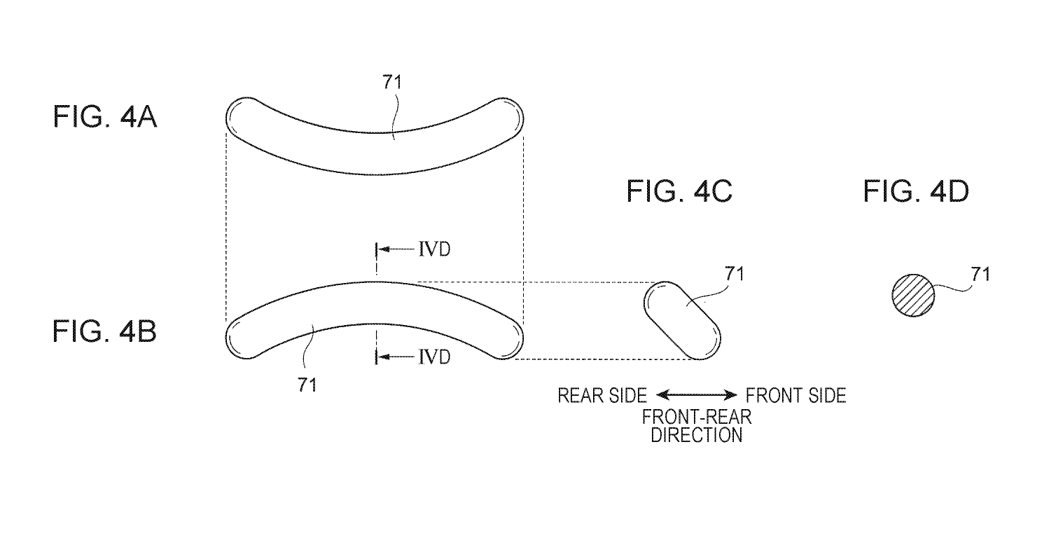

FIGS. 4A to 4D illustrate a detailed structure of the eyelid retainer 70. More specifically, FIG. 4A is a top view of an upper eyelid retaining member 71, FIG. 4B is a front view of the upper eyelid retaining member 71, FIG. 4C is a side view of the upper eyelid retaining member 71, and FIG. 4D is a sectional view of the upper eyelid retaining member 71 taken along line IVD-IVD of FIG. 4B.

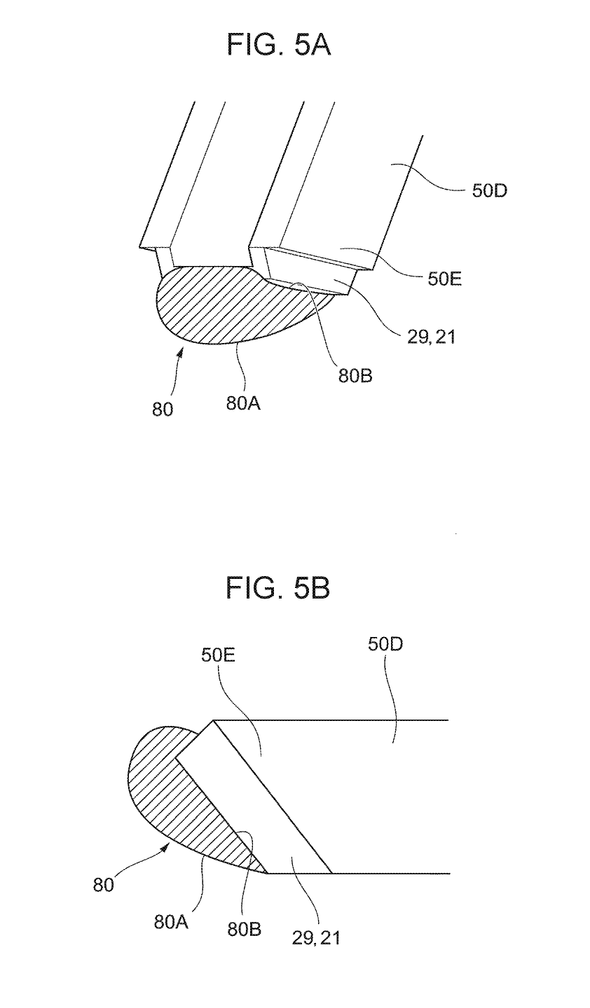

FIGS. 5A and 5B illustrate a detailed structure of the inner canthus presser 80. More specifically, FIG. 5A illustrates a structure of the inner canthus presser 80 and a region around the inner canthus presser 80 when seen in an arrow VA direction of FIG. 3A, and FIG. 5B illustrates a structure of the inner canthus presser 80 and a region around the inner canthus presser 80 when seen in an arrow VB direction of FIG. 3A.

Next, the eyelid retainer 70, the inner canthus presser 80, and disposition of the light emitting system 21 and the light receiving system 23 are described with reference to FIGS. 3A to 5B.

Initially, in order to measure the concentrations of the glucose and the like by detecting the light having passed through the aqueous humor by using the optical measuring apparatus 1, care should be taken to appropriately form the optical path 28 without, for example, refraction of the light in an unintended direction or blocking of the light by the eyelids of the subject or the like. Here, as a structure to avoid blocking of the optical path 28 by the eyelids of the subject, it is thought that the light emitting system 21 and the light receiving system 23 are disposed in a region Pa and a region Pb that are superposed on a white (sclera) of the eyeball 10 when seen from the front. However, with this structure, when the position of the light emitting system 21 or the light receiving system 23 deviates toward the rear side of the eyeball 10, the light emitting system 21 or the light receiving system 23 may be in contact with the white of the eyeball 10.

Accordingly, the optical measuring apparatus 1 according to the present exemplary embodiment is configured so that, even when the position of the light emitting system 21 or the light receiving system 23 deviates in the front-rear direction, contact of the light emitting system 21 or the light receiving system 23 with the white of the eyeball 10 is avoided and the optical path 28 is appropriately formed.

Specifically, the holding unit 50 holds the light emitting system 21 and the light receiving system 23 so that, when the eyeball 10 is seen from the front, the light emitting system 21 and the light receiving system 23 are positioned where the light emitting system 21 and the light receiving system 23 are superposed on near-inner-canthus skin 24A and near-outer-canthus skin 24E of FIG. 3B.

Furthermore, as illustrated in FIG. 3A, the optical measuring apparatus 1 according to the present exemplary embodiment includes the eyelid retainer 70 that retains the eyelids of the subject and the inner canthus presser 80 that presses the near-inner-canthus skin 24A of the subject.

The eyelid retainer 70 and the inner canthus presser 80 are provided at an end portion on the rear side of the holding unit 50. Here, the inner canthus presser 80 projects further to the rear side than the eyelid retainer 70. In more detail, in the illustrated example, the inner canthus presser 80 is disposed at a position that projects furthest to the rear side in the optical measuring apparatus 1.

The specific structures of the eyelid retainer 70 and the inner canthus presser 80 are sequentially described one by one below.

### The Structures of the Eyelid Retainer 70

Initially, the eyelid retainer 70 is described.

As illustrated in FIG. 3A, the eyelid retainer 70 includes the upper eyelid retaining member 71 and a lower eyelid retaining member 72. The upper eyelid retaining member 71 and the lower eyelid retaining member 72 are disposed on the upper side of the light emitting system 21 and the light receiving system 23 and on the lower side of the light emitting system 21 and the light receiving system 23, respectively. In other words, the upper eyelid retaining member 71 and the lower eyelid retaining member 72 face each other with the optical path 28 therebetween.

In order to improve the wearing feeling perceived by the subject, the eyelid retainer 70 is formed of a so-called elastic material such as, for example, silicon resin (silicone).

Here, as illustrated in FIG. 3A, the upper eyelid retaining member 71 and the lower eyelid retaining member 72 are supported by the holding unit 50. Specifically, the upper eyelid retaining member 71 and the lower eyelid retaining member 72 are supported by upper supports 50B and lower supports 50C that are secured to an end portion on the rear side of a cylindrical body 50A and extend along the optical path 28.

Additionally, when it is attempted to separately prepare and separately dispose the upper eyelid retaining member 71, the lower eyelid retaining member 72, the light emitting system 21, and the light receiving system 23 in a limited space around the eyeball 10 where the nose and eyelashes exist, these components are likely to interfere with one another. Thus, these components are integrally supported by the holding unit 50 as is the case with the illustrated example, so that the disposition of these components in the limited space is facilitated.

Furthermore, as illustrated in FIG. 3B, the upper eyelid retaining member 71 and the lower eyelid retaining member 72 are respectively provided at positions in the holding unit 50 facing the upper eyelid 18 and the lower eyelid 19. When the upper eyelid retaining member 71 and the lower eyelid retaining member 72 are pressed against the upper eyelid 18 and the lower eyelid 19, movements of the upper eyelid 18 and the lower eyelid 19 are restricted.

Next, the shapes of the upper eyelid retaining member 71 and the lower eyelid retaining member 72 are described with reference to FIGS. 4A to 4D. Here, although the upper eyelid retaining member 71 is described, the upper eyelid retaining member 71 and the lower eyelid retaining member 72 are symmetric about a plane the normal to which extends in the up-down direction (see FIG. 3B).

First, as illustrated in FIG. 4A, the upper eyelid retaining member 71 is a bar-shaped member (substantially cylindrical member) having a circular section (see FIG. 4D). Furthermore, an outer circumferential surface of the upper eyelid retaining member 71 in contact with the eyelid is formed to have a smoothly continuous curved surface without an edge formed thereon.

Furthermore, the upper eyelid retaining member 71 has a shape following the upper eyelid 18 (see FIG. 3B), that is, is curved along the eyeball 10 (see FIG. 3B). Specifically, as illustrated in FIGS. 4A to 4C, the upper eyelid retaining member 71 is curved so that a central portion thereof in the longitudinal direction projects forward and upward. As illustrated in FIG. 3A, the upper eyelid retaining member 71 and the lower eyelid retaining member 72 are curved so that the central portions thereof in the in-out direction are separated from each other.

### The Structure of the Inner Canthus Presser 80

Next, the inner canthus presser 80 is described.

First, as illustrated in FIG. 3A, the inner canthus presser 80 is supported by the holding unit 50. More specifically, the inner canthus presser 80 is secured to the light emitting system 21 (first mirror 29) held by a light-emitting-system holding unit 50D (to be described later). Here, the inner canthus presser 80 and the light-emitting-system holding unit 50D together with the eyelid retainer 70 are included in an example of a positioning device that positions the light emitting system 21 and the light receiving system 23 with respect to the eyeball 10. Additionally, the inner canthus presser 80, the light-emitting-system holding unit 50D, and the eyelid retainer 70 are supported by the holding unit 50 in positional relationships in which, when the optical measuring apparatus 1 is pressed against the eyeball 10, the light emitting system 21 (or the light receiving system 23) is disposed as intended relative to the eyeball 10. In order to improve the wearing feeling perceived by the subject, the inner canthus presser 80 is formed of a so-called elastic material such as, for example, silicon resin (silicone). In other words, the inner canthus presser 80 is formed of a softer material than the first mirror 29 or the light-emitting-system holding unit (holding unit) 50D.

The light-emitting-system holding unit 50D in the example of, for example, FIG. 3A has a substantially rectangular parallelepiped shape the longitudinal direction of which extends in the front-rear direction. The light-emitting-system holding unit 50D holds each of the optical components (light emitter 25, polarizer 27, and first mirror 29; see FIG. 1) of the light emitting system 21.

Furthermore, the light-emitting-system holding unit 50D includes a rear end portion (projection) 50E that projects further to the rear side than the upper eyelid retaining member 71 and the lower eyelid retaining member 72. The first mirror 29 is held at this rear end portion 50E.

The inner canthus presser 80 is secured to a surface of the first mirror 29 on the rear side by a known securing method such as a method using an adhesive (not illustrated). Additionally, the first mirror 29 of, for example, FIG. 3A is pressed against the subject through the inner canthus presser 80.

Here, the inner canthus presser 80 is integrated with the first mirror 29 which is an optical component positioned at a rearmost position of the light emitting system 21.

Disposition of the components in the limited space may be facilitated by providing the inner canthus presser 80 in the first mirror 29 as described above. In more detail, compared to a structure in which the inner canthus presser 80 and the first mirror 29 are separately disposed, disposition of the first mirror 29 at a position further to the rear side may be facilitated. Furthermore, compared to the structure in which the inner canthus presser 80 and the first mirror 29 are separately disposed, the occurrences of a situation in which the positioning of the optical measuring apparatus 1 (see FIG. 3A) is obstructed due to contact of the first mirror 29 (light-emitting-system holding unit 50D) with the nose may be suppressed. Furthermore, when the inner canthus presser 80 is pressed against the skin, positioning of the first mirror 29 may also be simultaneously performed.

Here, disposition of the inner canthus presser 80 is further described.

Referring to FIG. 3B, the inner canthus presser 80 is provided at a position, in the up-down direction, between the upper eyelid retaining member 71 and the lower eyelid retaining member 72 and facing the inner canthus sides of the upper eyelid 18 and the lower eyelid 19. Here, the inner canthus sides of the upper eyelid 18 and the lower eyelid 19 refer to portions of the upper eyelid 18 and the lower eyelid 19 on the inner side (nose side) relative to a pupil 15. Furthermore, when seen from a different viewpoint, the inner canthus presser 80 is positioned in a region of the near-inner-canthus skin 24A. Here, the term “near-inner-canthus skin” refers to a portion of skin existing in a region on the inner side (nose side) relative to the pupil 15 where the skin is able to be pressed toward an orbit 17 (to be described later) and, when pressed, able to be pressed further to the rear side than the position of the front end of the eyeball 10. Furthermore, the term “near-outer-canthus skin” refers to a portion of skin existing in both the region where the skin is able to be pressed toward the orbit 17 and a region where the skin is not able to be pressed toward the orbit 17 on the outer side (ear side) relative to the pupil 15.

In more detail, the inner canthus presser 80 is positioned, for example, in a region on the inner canthus sides of the upper eyelid 18 and the lower eyelid 19 and closer (outer side) to the eyeball 10 than an inner circumferential surface 17A of the orbit 17. When seen from a different viewpoint, the inner canthus presser 80 is positioned so as to be brought into contact with the near-inner-canthus skin 24A which is substantially at the same position (level) as that of an inner corner of the eye 18A (inner canthus) in the up-down direction of the eyeball 10. The position of the inner corner of the eye (inner canthus) 18A in the up-down direction of the eyeball 10 is a rearmost (rear side of the eyeball 10) position of the skin out of the near-inner-canthus skin 24A. At this portion, compared to positioning at a different position, the depth to which the skin is to be pressed is reduced. The term substantially the same position (level) as that of the inner corner of the eye 18A refers to a range ±1 mm from the position of the inner corner of the eye 18A in the up-down direction of the eyeball 10.

Meanwhile, at the position of the inner corner of the eye 18A in the up-down direction, the distance between the inner corner of the eye 18A and the inner circumferential surface 17A of the orbit 17 is smallest. Thus, the region for positioning may be difficult to allocate depending on the shape of the inner canthus presser 80. In such a case, the inner canthus presser 80 may be positioned so that the tip of the inner canthus presser 80 is brought into contact with a position shifted upward or downward (for example, an upper position 24C or a lower position 24D) from the position of the inner corner of the eye 18A. As the tip of the inner canthus presser 80 is shifted from the position of the inner corner of the eye 18A in the up-down direction, the distance between the inner corner of the eye 18A and the inner circumferential surface 17A of the orbit 17 increases, and accordingly, a region in which the inner canthus presser 80 is able to be positioned increases in size.

It is noted that where to position the inner canthus presser 80 in the region of the near-inner-canthus skin 24A is not necessarily fixed. The optical measuring apparatus 1 may be configured so that the inner canthus presser 80 is positioned so as to facilitate the formation of the optical path 28 on a subject-by-subject basis by considering, for example, the shape of the region around the eyeball 10 of the subject, the shape of the inner canthus presser 80, and accuracy of the positioning.

Furthermore, although the details will be described later, when the inner canthus presser 80 is pressed against the near-inner-canthus skin 24A, the near-inner-canthus skin 24A is pressed toward the rear side of the orbit 17.

Next, the shape of the inner canthus presser 80 is described with reference to FIGS. 5A and 5B.

As illustrated in FIGS. 5A and 5B, the inner canthus presser 80 has a substantially semispherical shape. In other words, the inner canthus presser 80 includes a convex portion 80A that projects rearward. The inner canthus presser 80 also includes a concave portion 80B that follows the shape of the first mirror 29 disposed on a side (front side) in contact with the first mirror 29 (see FIG. 3A).

The convex portion 80A is brought into contact with the eyelids. The convex portion 80A in an example of, for example, FIG. 3A has a smoothly continuous curved surface without an edge.

### Operations of the Eyelid Retainer 70 and the Inner Canthus Presser 80

FIG. 6 illustrates operations of the eyelid retainer 70 and the inner canthus presser 80. In FIG. 6, a section at a central position of the eyeball 10 in the up-down direction is seen from a head side (upper side) of the subject.

Next, operations of the eyelid retainer 70 and the inner canthus presser 80 are described with reference to FIGS. 3A to 5B.

Here, for convenience of description, the structure of the eyeball 10 and a region around the eyeball 10 is described, the positional relationship between the eyeball 10 and the optical path 28 is described, and after that, the specific operations of the eyelid retainer 70 and the inner canthus presser 80 are described.

### The Structure of the Eyeball 10 and the Region Around the Eyeball 10

Initially, the structure of the Eyeball 10 and the region around the Eyeball 10 is described.

As illustrated in FIG. 6, the eyeball 10 has a substantially spherical external shape and has a vitreous body 11 at the center. The crystalline lens 12 functioning as a lens is embedded in part of the vitreous body 11. The anterior chamber 13 exists on the front side of the crystalline lens 12, and the cornea 14 exists on the front side of the anterior chamber 13. The periphery the crystalline lens 12 is surrounded by an iris, and the pupil 15 exists at the center of the crystalline lens 12. The vitreous body 11 except for a portion in contact with the crystalline lens 12 is covered by the retina 16.

The anterior chamber 13 is a region that is surrounded by the cornea 14 and the crystalline lens 12 and that projects in a convex shape from the spherical shape of the eyeball 10. This anterior chamber 13 has a circular shape when seen from the front. The anterior chamber 13 is filled with the aqueous humor.

The eyeball 10 is contained in the orbit 17, which is a depression (recess) in the bone of skull. The eyeball 10 is covered with the eyelids (upper eyelid 18 and lower eyelid 19).

Here, the orbit 17 referred to in the present exemplary embodiment means, as illustrated in FIG. 6, a region 17B that includes a region from which the bone of skull (inner circumferential surface 17A of the orbit 17) starts to be depressed toward the rear side of the eyeball 10 relative to the surface of the skin. A region 17C and a region 17D, in which the distance between the surface of the skin and the inner circumferential surface 17A of the orbit 17 gradually increases, exist on the inner canthus side and the outer canthus side of the region 17B of the orbit 17. That is, the amount of the near-inner-canthus skin 24A and the amount of the near-outer-canthus skin 24E pressed toward the rear side of the eyeball 10 are larger in the region 17C on the inner canthus side and the region 17D on the outer canthus side in the region 17B of the orbit 17 than in a region other than the region 17B of the orbit 17.

Furthermore, as illustrated in FIG. 6, in a general subject, the near-inner-canthus skin 24A is positioned further to the front side than the near-outer-canthus skin 24E. Thus, in order to position the first mirror 29 (light emitting system 21) and the second mirror 31 (light receiving system 23) when the eyeball 10 faces the front (normal vision state), it is required that the near-inner-canthus skin 24A be pressed for some subjects.

Thus, according to the present exemplary embodiment, the first mirror 29 (light emitting system 21) is pressed toward the orbit 17 by utilizing the fact that the amount of the portion of skin able to be pressed toward the rear side of the orbit 17 is larger in the region 17C on the inner canthus side in the region 17B of the orbit 17 than in a region other than the region 17B of the orbit 17. In other words, the first mirror 29 (light emitting system 21) is positioned at a position reached by the near-inner-canthus skin 24A having been pressed toward a portion between the inner circumferential surface 17A of the orbit 17 and the eyeball 10. Thus, when measurement is performed while the eyeball 10 faces the front (normal vision state), even in the case where a space in which the first mirror 29 (light emitting system 21) is disposed does not exist (is small) due to the near-inner-canthus skin 24A, the optical path 28 crossing the anterior chamber 13 is provided by pressing the near-inner-canthus skin 24A into the orbit 17.

### The Positional Relationship Between the Eyeball 10 and the Optical Path 28

Next, the positional relationship between the eyeball 10 and the optical path 28 of the optical system 20 is described.

As illustrated in FIG. 6, the light output from the light emitting system 21 is incident upon the anterior chamber 13 in a direction directed outward in the in-out direction and directed forward in the front-rear direction. Furthermore, the light having passed through the anterior chamber 13 is incident upon the light receiving system 23 in a direction directed outward in the in-out direction and directed rearward in the front-rear direction.

That is, the light emitting system 21 (first mirror 29) is disposed so that the light output from the light emitting system 21 toward the anterior chamber 13 obliquely advances forward in the front-rear direction. In other words, the first mirror 29 is disposed further to the rear side than a front top portion of an exposed portion (anterior chamber 13) of the eyeball 10.

Furthermore, the light receiving system 23 (second mirror 31) is disposed so as to receive the light that obliquely advances rearward from the anterior chamber 13 in the front-rear direction. In other words, the second mirror 31 is disposed further to the rear side than the front top portion of the exposed portion (anterior chamber 13) of the eyeball 10.

The reason for this disposition is as follows. That is, the light output from the light emitter 25 passes through the cornea 14 and is incident upon the anterior chamber 13. At this time, the refractive index of the aqueous humor in the cornea 14 and the anterior chamber 13 (n=about 1.37) is larger than that of air (n=about 1.0), and the cornea 14 and the anterior chamber 13 have convex shapes. Thus, the optical path 28 is bent rearward (to eyeball 10 side). Furthermore, even after having passed through the anterior chamber 13, the optical path 28 is further bent rearward. Thus, the light emitting system 21 and the light receiving system 23 are disposed on the basis of the fact that the optical path 28 is bent rearward due to the passage through the anterior chamber 13.

Furthermore, there is a small space for setting the optical system 20 around the eye (eyeball 10) of a face where the nose (bridge of nose) exists. Furthermore, when the light is directed out of the anterior chamber 13, correct measurement is not able to be performed. Accordingly, the optical path 28 may be set so as to cross the anterior chamber 13 without being directed out of the anterior chamber 13.

Furthermore, the optical rotation α_{M} is affected by the optical path length that is a length by which the light passes through the aqueous humor in the anterior chamber 13. Thus, in order to suppress variation of the optical path length, the optical path 28 may be set as described above. In the optical measuring apparatus 1 of, for example, FIG. 1, a large optical path length is able to be set due to the setting of the optical path 28 that crosses the anterior chamber 13.

### Specific Operations of the Eyelid Retainer 70 and the Inner Canthus Presser 80

Next, operations of the eyelid retainer 70 and the inner canthus presser 80 are specifically described.

Initially, the near-inner-canthus skin 24A is pressed rearward by pressing the inner canthus presser 80 against the eyelids (upper eyelid 18 and lower eyelid 19) of the subject. In more detail, the inner canthus presser 80 presses the near-inner-canthus skin 24A toward the portion between the inner circumferential surface 17A of the orbit 17 and the eyeball 10.

As illustrated in FIG. 6, the length of a portion of the eyeball 10 that projects relative to the near-inner-canthus skin 24A when the near-inner-canthus skin 24A is not pressed, that is, a degree of projection Ga of the eyeball 10 relative to the near-inner-canthus skin 24A is about 6 mm. A degree of projection Gb relative to the near-outer-canthus skin 24E is about 11 to 12 mm. A movement amount Gc of the near-inner-canthus skin 24A when the near-inner-canthus skin 24A is pressed by the inner canthus presser 80 is, for example, 3 to 5 mm on condition that the subject does not perceive pain. That is, the sum of the degree of projection Ga relative the near-inner-canthus skin 24A when the near-inner-canthus skin 24A is pressed and the movement amount Gc is the same as or substantially the same as the degree of projection Gb relative to the near-outer-canthus skin 24E.

As the inner canthus presser 80 presses the near-inner-canthus skin 24A as described above, the size of the space for disposing the first mirror 29 (light emitting system 21) is increased. That is, the first mirror 29 is able to be disposed further to the rear side. This facilitates the formation of the optical path 28 passing through the aqueous humor in the anterior chamber 13 even when the eyeball 10 faces the front (normal vision state). In other words, blocking of the optical path 28 by the upper eyelid 18 and the lower eyelid 19 is suppressed.

The position of the optical measuring apparatus 1 (see FIG. 1) attached to the subject is determined by pressing the eyelid retainer 70 (upper eyelid retaining member 71 and lower eyelid retaining member 72) against the eyelids of the subject (upper eyelid 18 and lower eyelid 19). That is, the eyelid retainer 70 together with the inner canthus presser 80, the light-emitting-system holding unit 50D, and so forth functions as the positioning device with respect to the eyeball 10. Furthermore, by pressing the eyelid retainer 70, stress acts in directions in which the upper eyelid 18 and the lower eyelid 19 open along the eyeball 10, thereby keeping the upper eyelid 18 and the lower eyelid 19 open.

Here, as the near-inner-canthus skin 24A is pressed by the inner canthus presser 80, forces to close the eyelids act. In other words, as the eyelids are pressed rearward by the inner canthus presser 80, stress acts on the eyelids continuous with the near-inner-canthus skin 24A so as to close the eyelids. For this, the eyelid retainer 70 suppresses the forces to close the eyelids. Thus, the eyelids are kept open. In other words, when the near-inner-canthus skin 24A is pressed toward the inside of the orbit 17, the eyelids are moved in closing directions due to the pressing, thereby reducing a region of the eyeball 10 exposed from the skin. This may restrict the optical path 28 passing through the aqueous humor in the anterior chamber 13. According to the present exemplary embodiment, the eyelid retainer 70 suppresses the reduction of the exposed region of the eyeball 10. Thus, compared to a structure without the eyelid retainer 70, formation of the optical path 28 passing through the aqueous humor in the anterior chamber 13 may be facilitated. The term “suppresses the reduction of the exposed region of the eyeball 10” means that, compared to the structure without the eyelid retainer 70, the exposed region of the eyeball 10 is increased at the time when the light emitting system 21 outputs the light. This includes a state in which the exposed region of the eyeball 10 is larger than that in a state in which the skin is not pressed.

### Measurement of Adduction

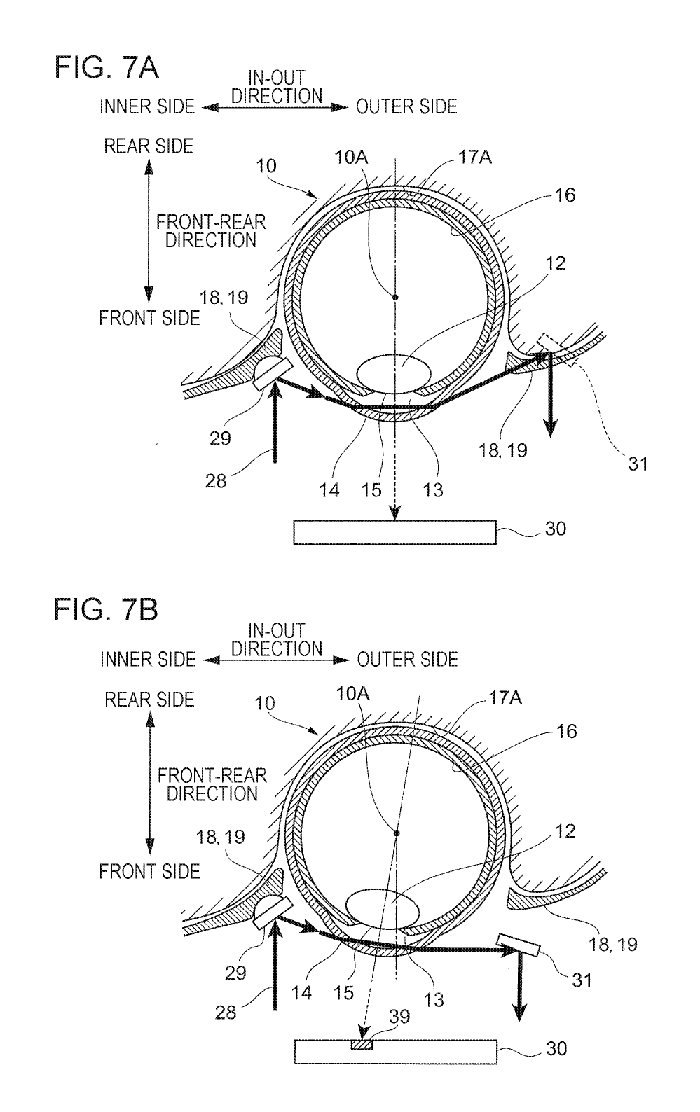

Referring to FIGS. 7A and 7B, an example of measurement with the eyeball 10 adducted is described. Here, FIG. 7A illustrates the disposition of the optical measuring apparatus 1 when measurement is performed in the normal vision state. The difference in state between FIG. 6 and FIG. 7A is that, in the state of FIG. 7A, the near-outer-canthus skin 24E moves forward in the front-rear direction compared to that in the shape of the face of FIG. 6. FIG. 7B illustrates the disposition of the optical measuring apparatus 1 when measurement is performed with the eyeball 10 adducted. For convenience of drawing, the dimensions of FIGS. 7A and 7B are different from those of FIG. 6.

The term “adduction” refers to rotation of the eyeball 10 (pupil 15) to the inner canthus side (nose side) in the in-out direction within a range of ±45° in the up-down direction when the eyeball 10 is seen from the front, and the term “abduction” refers to rotation of the eyeball 10 (pupil 15) to the outer canthus side (ear side) in the in-out direction within a range of ±45° in the up-down direction when the eyeball 10 is seen from the front. Here, rotation of the eyeball 10 about an axis 10A to the inner side (nose side) is an example of the “adduction”, and rotation of the eyeball 10 about the axis 10A to the outer side (ear side) is an example of the “abduction”.

First, as illustrated in FIG. 7A, the near-outer-canthus skin 24E may project further to the front side in the front-rear direction than that in the shape of FIG. 6 depending on the shape of the face of the subject. For example, when the positions of the near-inner-canthus skin 24A and the near-outer-canthus skin 24E in the front-rear direction are the same or substantially the same, it may be difficult, in the normal vision state, to form the optical path 28 passing through the aqueous humor in the anterior chamber 13 unless the skin on the outer canthus side is pressed as much as that on the inner canthus side.

Furthermore, subjects generally have a larger number of eyelashes on the outer canthus side than on the inner canthus side. Thus, even when the near-outer-canthus skin 24E is positioned further to the rear side in the front-rear direction than the near-inner-canthus skin 24A as in the shape of FIG. 6, it may be difficult, in the normal vision state, to form the optical path 28 passing through the aqueous humor in the anterior chamber 13 due to obstruction caused by the eyelashes on the outer canthus side.

Thus, as illustrated in FIG. 7B, in addition to the pressing of the near-inner-canthus skin 24A, the eyeball 10 is adducted. This allows the position of the light receiving system 23 (second mirror 31) on the outer canthus side to be disposed further to the front side in the front-rear direction of the eyeball 10 than that in the normal vision state. That is, the light receiving system 23 (second mirror 31) is able to be disposed away from the near-outer-canthus skin 24E and the eyelashes on the outer canthus side to the front side in the front-rear direction. Thus, the blocking of the optical path 28 by the near-outer-canthus skin 24E and the eyelashes on the outer canthus side may be suppressed. This may facilitate the formation of the optical path 28 passing through the aqueous humor in the anterior chamber 13.

Specifically, as illustrated in FIG. 7B, the mark 39 is displayed in the display unit 30 of the optical measuring apparatus 1 so as to adduct the eyeball 10 during the measurement. More specifically, the mark 39 is displayed at such a position that the eyeball 10 is adducted when the mark 39 is visually recognized by the subject. The line of sight is guided to be directed to the mark 39 by, for example, displaying the mark 39 in the display unit 30 as described above. Thus, the eyeball 10 is adducted, and the measurement is performed in a state in which the optical path 28 is formed.

In the example of, for example, FIG. 7B, the second mirror 31 is disposed further to the front side than the first mirror 29. This suppresses the light receiving system 23 (second mirror 31) to be brought into contact with the subject. Additionally, pressing of the subject by the light receiving system 23 (second mirror 31) is avoided, thereby the attaching feeling perceived by the subject may be improved.

Furthermore, although the mark 39 is displayed in the display unit 30 during the measurement here, the mark 39 may be constantly displayed. In this case, the display unit 30 does not necessarily include the display device that electronically displays an image. Alternatively, a member or a shape able to function as the mark 39 may be provided.

### Second Exemplary Embodiment

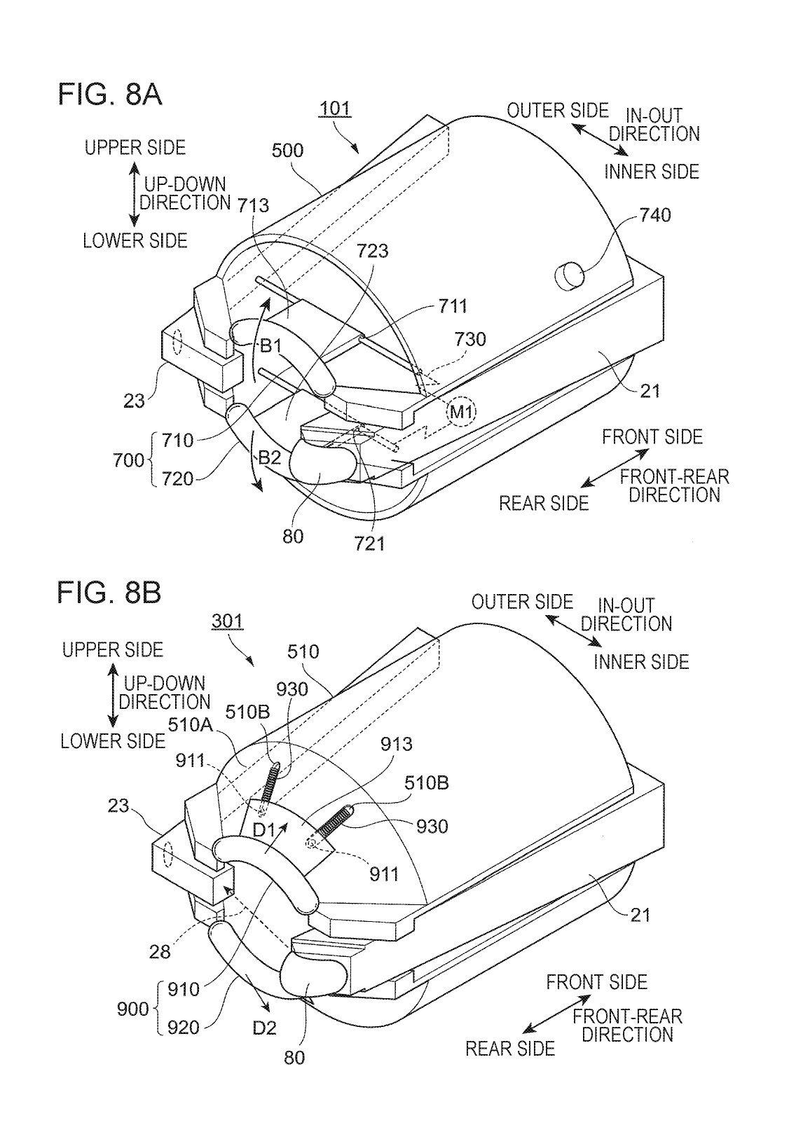

FIGS. 8A and 8B illustrate structures of an optical measuring apparatus 101 and an optical measuring apparatus 301 according to other exemplary embodiments. More specifically, FIG. 8A illustrates the structure of the optical measuring apparatus 101 according to a second exemplary embodiment, and FIG. 8B illustrates the structure of the optical measuring apparatus 301 according to a third exemplary embodiment.

Although the position of the eyelid retainer 70 is fixed in the optical measuring apparatus 1 of, for example, FIG. 1 referred to in the above description, this is not limiting. For example, an eyelid retainer 700 (upper eyelid retaining member 710 and lower eyelid retaining member 720) may be movable as those of the optical measuring apparatus 101 of FIG. 8A.

Specifically, the optical measuring apparatus 101 of FIG. 8A includes a motor M1, a gear group 730 that transmits a drive force from the motor M1, rotational shafts 711 and 721 that extend in the in-out direction, connecting members 713 and 723 respectively connected to the upper eyelid retaining member 710 and the lower eyelid retaining member 720. The motor M1, the gear group 730, the rotational shafts 711 and 721, and the connecting members 713 and 723 are disposed in a holding unit 500. The optical measuring apparatus 101 also includes an operating button 740 that triggers driving of the motor M1.

Operation of this optical measuring apparatus 101 is described.

Initially, the optical measuring apparatus 101 is attached to the eyelids of the subject, and the upper eyelid retaining member 710 and the lower eyelid retaining member 720 are brought into contact with the upper eyelid 18 and the lower eyelid 19 (see FIG. 3B). In this state, when, for example, the subject operates the operating button 740, the motor M1 is driven. As the motor M1 is driven, the upper eyelid retaining member 710 and the lower eyelid retaining member 720 are moved so as to be separated from each other (see arrows B1 and B2). This causes the upper eyelid 18 and the lower eyelid 19 to be opened. Thus, with the optical measuring apparatus 101, the eyelids may be more reliably opened by driving the motor M1.

### Third Exemplary Embodiment

Alternatively, as illustrated in FIG. 8B, an eyelid retainer 900 (upper eyelid retaining member 910 and lower eyelid retaining member 920) may be moved by a force with which the optical measuring apparatus 301 is pressed against the subject.

Specifically, the optical measuring apparatus 301 includes the following structure as a mechanism that moves the upper eyelid retaining member 910. That is, the optical measuring apparatus 301 includes a truncated cone-shaped covering surface 510A that covers the rear side of the holding unit 510 and guide grooves 510B provided along an outer circumferential surface of the covering surface 510A. The longitudinal direction of the guide grooves 510B extend in the up-down direction. The optical measuring apparatus 301 also includes pin-shaped guided portions 911 movable in the guide grooves 510B, a connecting member 913 that connects the guided portions 911 and the upper eyelid retaining member 910 to one another, and springs 930 that urge the connecting member 913. Here, the springs 930 urge the connecting member 913 in directions in which the upper eyelid retaining member 910 and the lower eyelid retaining member 920 approach each other.

Although illustration of it is omitted from FIG. 8B, the optical measuring apparatus 301 includes a mechanism that moves the lower eyelid retaining member 920 similarly to or in the same way as the mechanism that moves the upper eyelid retaining member 910.

Operation of this optical measuring apparatus 301 is described.

Initially, the optical measuring apparatus 301 is attached to the eyelids of the subject. At this time, the upper eyelid retaining member 910 and the lower eyelid retaining member 920 are brought into contact with the upper eyelid 18 and the lower eyelid 19 (see FIG. 3B).

When, for example, the subject applies a force with which the optical measuring apparatus 301 is further pressed against the upper eyelid 18 and the lower eyelid 19, the guided portions 911 are moved in the guide grooves 510B while opposing the urging forces applied by the springs 930. This causes the lower eyelid retaining member 920 and the upper eyelid retaining member 910 connected to the connecting member 913 to move in directions in which the upper eyelid retaining member 910 and the lower eyelid retaining member 920 are separated from each other (see arrows D1 and D2). As a result, the upper eyelid 18 and the lower eyelid 19 are opened.

Thus, with the optical measuring apparatus 301, the eyelids may be reliably opened by utilizing the force applied by the subject to press the optical measuring apparatus 301 against the upper eyelid 18 and the lower eyelid 19 without receiving a drive force from a drive source.

### Fourth Exemplary Embodiment

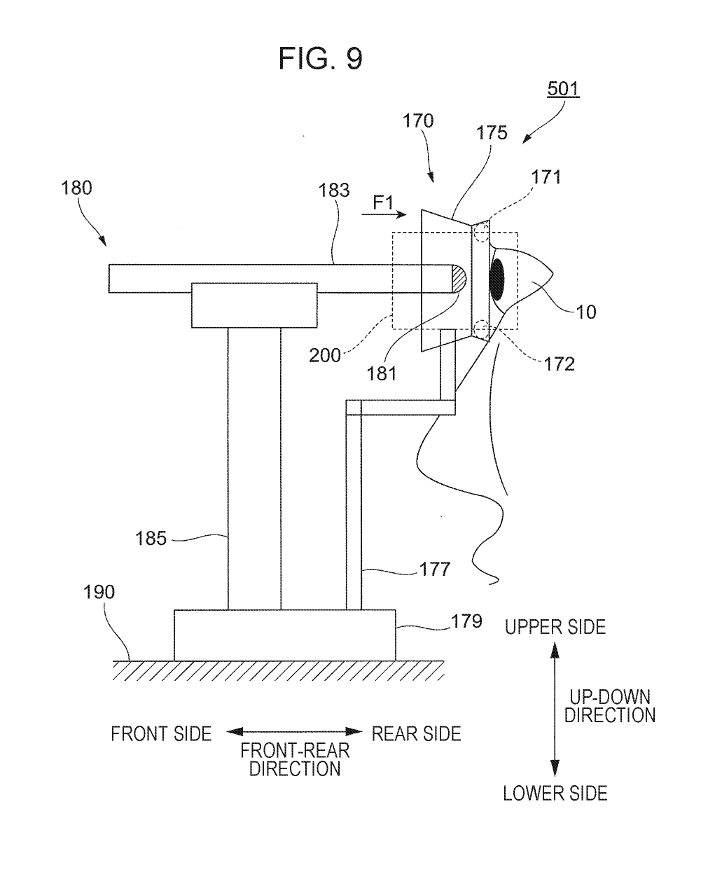

FIG. 9 illustrates a structure of an optical measuring apparatus 501 according to a fourth exemplary embodiment.

Although the position of the inner canthus presser 80 is fixed in the optical measuring apparatus 1 of, for example, FIG. 1 referred to in the above description, this is not limiting. For example, the inner canthus presser 80 may be movable as that of the optical measuring apparatus 501 of FIG. 9.

Specifically, the optical measuring apparatus 501 of FIG. 9 includes an eyelid retainer 170, a movable presser 180, and an optical system 200. The eyelid retainer 170 is brought into contact with the eyelids of the subject so as to retain the eyelids. The movable presser 180 presses the inner canthus side of the eyelids of the subject. The optical system 200 is used to measure the characteristics of the aqueous humor in the eyeball 10 of the subject similarly to or in the same way as that with the optical measuring apparatus 1 of, for example, FIG. 1.

The eyelid retainer 170 includes an upper eyelid retaining member 171, a lower eyelid retaining member 172, a holding member 175 that holds the upper eyelid retaining member 171 and the lower eyelid retaining member 172, a support member 177 that supports the holding member 175, and a base 179 that supports the support member 177.

Furthermore, the movable presser 180 includes an inner canthus presser 181, a movement unit 183, and a slide support unit 185. The inner canthus presser 181 presses the near-inner-canthus skin 24A of the subject similarly to or in the same way as that with the optical measuring apparatus 1 of, for example, FIG. 1. The movement unit 183, in which the inner canthus presser 181 is provided, is moved in the front-rear direction. The movement unit 183 is slidably supported by the slide support unit 185. The movement unit 183 in this example is moved in the front-rear direction by receiving drive from a motor (not illustrated). The slide support unit 185 is secured to the base 179.

Furthermore, the optical system 200 of this example is secured to the movement unit 183 of the movable presser 180. The optical system 200 is moved in the front-rear direction together with the movement unit 183.

Next, operation of this optical measuring apparatus 501 is described.

Initially, the optical measuring apparatus 501 is secured to a workbench 190 or the like. The subject presses his or her face against the optical measuring apparatus 501 at a position where the eyelids of the subject are brought into contact with the upper eyelid retaining member 171 and the lower eyelid retaining member 172 of the optical measuring apparatus 501. In this state, when, for example, an operating button (not illustrated) is operated, the motor (not illustrated) is driven.

As the motor is driven, the slide support unit 185 is moved rearward in the front-rear direction (see arrow F1). Thus, the inner canthus presser 181 attached to the end of the slide support unit 185 presses the inner canthus side of the eyelids of the subject rearward in the front-rear direction. As a result, the optical path 28 (see FIG. 1) passing through the aqueous humor may be more reliably formed. In this state, measurement of the characteristics of the aqueous humor in the eyeball 10 is performed by the optical system 200.

Although the optical system 200 here is secured to the movement unit 183 of the movable presser 180, this is not limiting. For example, the optical system 200 may be driven independently of the movement unit 183 of the movable presser 180 so as to be moved in the front-rear direction. Alternatively, the position of the optical system 200 may be fixed.

Furthermore, although the movement unit 183 here is moved by receiving the drive force from the motor (not illustrated), this is not limiting. For example, the movement unit 183 may be manually moved by a measurer or the like who operates the optical measuring apparatus 501.

Furthermore, although the movable presser 180 here is provided in the optical measuring apparatus 501 secured to the workbench 190 or the like, this is not limiting. For example, the movable presser 180 may be provided in the optical measuring apparatus 1 as illustrated in, for example, FIG. 1. That is, the inner canthus presser 80 (FIG. 1) provided in the body 50A (see FIG. 1) may be moved in the front-rear direction.

### Variations

Although the inner canthus presser 80 presses (applies pressure to) the near-inner-canthus skin 24A according to the above description, this is not limiting. For example, only the near-outer-canthus skin 24E may be pressed or both the near-inner-canthus skin 24A and the near-outer-canthus skin 24E may be pressed. In order to press the near-outer-canthus skin 24E into the orbit 17, it is sufficient that a member that is the same as or similar to the inner canthus presser 80 be provided at an end of the light receiving system 23.

Furthermore, although the inner canthus presser 80 includes a single member according to the above description, this is not limiting. The inner canthus presser 80 may include plural members. In more detail, the inner canthus presser 80 may include, for example, two members which respectively press the upper eyelid 18 and the lower eyelid 19 included in the near-inner-canthus skin 24A.

Furthermore, the shape of the inner canthus presser 80 is not particularly limited. In more detail, the inner canthus presser 80, of course, may have any other shape such as a spherical shape, an arc shape, or a plate shape as long as the inner canthus presser 80 is able to be brought into contact with the near-inner-canthus skin 24A and maintain a state in which a gap having such a size that allows the light crossing the eyeball 10 to pass through the gap is formed.

Furthermore, the position where the inner canthus presser 80 is provided in the optical measuring apparatus 1 is not particularly limited. In more detail, the inner canthus presser 80 may be held by the light-emitting-system holding unit 50D or secured to the first mirror 29 with another member interposed therebetween as long as the inner canthus presser 80 is able to press the near-inner-canthus skin 24A rearward.

Furthermore, although the eyelid retainer 70 includes plural members (upper eyelid retaining member 71 and lower eyelid retaining member 72) according to the above description, this is not limiting. For example, the eyelid retainer 70 may include either the upper eyelid retaining member 71 or the lower eyelid retaining member 72. Alternatively, the upper eyelid retaining member 71 and the lower eyelid retaining member 72 may be integrally formed with each other.

Furthermore, the upper eyelid retaining member 71 and the lower eyelid retaining member 72 may have shapes different from those in the above description. In more detail, each of the upper eyelid retaining member 71 and the lower eyelid retaining member 72, of course, may have any other shape such as a semispherical shape or a plate shape as long as one of the upper eyelid retaining member 71 and the lower eyelid retaining member 72 is able to be brought into contact with at least one of the upper eyelid 18 and the lower eyelid 19 (see FIG. 3B), and as long as the upper eyelid retaining member 71 and the lower eyelid retaining member 72 are able to maintain a state in which a gap having such a size that allows the light to pass through the gap is formed between the upper eyelid 18 and the lower eyelid 19.

Alternatively, a structure in which one of the upper eyelid retaining member 71 and the lower eyelid retaining member 72 is moved as described with reference to FIG. 9 and the other of the upper eyelid retaining member 71 and the lower eyelid retaining member 72 is fixed is possible. For example, it is possible that the upper eyelid retaining member 71 is movable and the lower eyelid retaining member 72 is fixed.

Although the eyelid retainer 70 (upper eyelid retaining member 71 and lower eyelid retaining member 72) directly retains the eyelids of the subject, this is not limiting as long as the eyelid retainer 70 is brought into contact with the skin near the eyeball 10 or the like of the subject so as to keep the eyelids of the subject open.

The skin near the eyeball 10 is a region as follows: that is, when this region is brought into contact with the inner canthus presser 80 and the eyelid retainer 70, a movement (opening/closing) of at least one of the upper eyelid 18 and the lower eyelid 19 is restricted.

Furthermore, although the inner canthus presser 80 and the eyelid retainer 70 are formed of silicon resin (silicone) according to the above description, this is not limiting. For example, the inner canthus presser 80 and the eyelid retainer 70 may be formed of a material such as metal or resin other than silicon resin. Alternatively, the inner canthus presser 80 and the eyelid retainer 70 may be formed of a resin member made of vinyl chloride resin or the like which is, for example, coated with an acrylic adhesive. Furthermore, for example, medical adhesive tape may be provided on outer circumferential surfaces of the inner canthus presser 80 and the eyelid retainer 70.

The inner canthus presser 80 and the eyelid retainer 70 may be formed of a material having a high friction and high safety.

Although the light emitting system 21 is disposed on the nose side (inner canthus side) and the light receiving system 23 is disposed on the ear side (outer canthus side) according to the above description, a reverse structure is possible, that is, the light emitting system 21 may be disposed on the ear side and the light receiving system 23 may be disposed on the nose side.

Furthermore, the optical path 28 is not limited to that illustrated in, for example, FIG. 1. It is sufficient that the light output from the light emitter 25 pass through the anterior chamber 13 so as to cross the anterior chamber 13 and be received by the light receiver 35. The above-described passage of the light through the anterior chamber 13 so as to cross the anterior chamber 13 refers to passage of the light, when the eyeball 10 is seen from the front, in a path at an angle following the in-out direction rather than in a path at an angle following the up-down direction (that is, in a range smaller than ±45° relative to the horizontal axis in the in-out direction), including the passage of the light obliquely in the front-rear direction.

Furthermore, although the light emitting system 21 disposed on the nose side projects further to the front side than the light receiving system 23 disposed on the ear side according to the above description, this is not limiting. For example, the light emitting system 21 and the light receiving system 23 may be disposed at positions corresponding to each other (the same position) in the front-rear direction or the light receiving system 23 disposed on the ear side may project further to the front side than the light emitting system 21 disposed on the nose side.

Furthermore, although the method of calculating the concentrations of the target optically active substances contained in the aqueous humor has been described, other characteristics of the aqueous humor may be measured.

Furthermore, the structure described according to the exemplary embodiments herein may be applied so as to obtain the characteristics of the cornea and so forth existing in the optical path 28 in addition to the characteristics of the aqueous humor. That is, the structure described according to the exemplary embodiments herein may be applied to a device as long as this device causes the light to be incident upon the eyeball 10 from the outside of the eyeball 10 and pass through the cornea 14 and the aqueous humor in the anterior chamber 13 and receives the light having passed through the cornea 14 and the aqueous humor.

Furthermore, although the eyeball 10 is that of the left eye in the description according to the exemplary embodiments herein, of course, the optical measuring apparatus 1 may be applied to the right eye (not illustrated).

Although the various exemplary embodiments and the variations have been described, of course, these exemplary embodiments and variations may be combined to one another.

Furthermore, the present disclosure is not limited to the above-described exemplary embodiments and may be embodied in various forms without departing from the gist of the present disclosure.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An optical measuring apparatus comprising:
a light output device that outputs light so as to cross an anterior chamber of an eyeball of a subject;
a light receiving device that receives the light having crossed the anterior chamber; and
a positioning device that positions the light output device and the light receiving device at such positions that, when the eyeball is adducted, the light output from the light output device crosses the anterior chamber and is received by the light receiving device.

2. The optical measuring apparatus according to claim 1,
wherein the positioning device positions one of the light output device and the light receiving device disposed on an inner canthus side of the eyeball at a position reached by the one of the light output device and the light receiving device having been pressed into an orbit containing the eyeball.

3. The optical measuring apparatus according to claim 1,
wherein the positioning device positions one of the light output device and the light receiving device disposed on an outer canthus side further to a front side of the eyeball than another of the light output device and the light receiving device disposed on an inner canthus side.

4. The optical measuring apparatus according to claim 2,
wherein the positioning device positions one of the light output device and the light receiving device disposed on an outer canthus side further to a front side of the eyeball than another of the light output device and the light receiving device disposed on the inner canthus side.

5. The optical measuring apparatus according to claim 1, further comprising:
a display that displays a mark at such a position that the eyeball is adducted when the subject visually recognizes the mark.

6. The optical measuring apparatus according to claim 2, further comprising:
a display that displays a mark at such a position that the eyeball is adducted when the subject visually recognizes the mark.

7. The optical measuring apparatus according to claim 3, further comprising:
a display that displays a mark at such a position that the eyeball is adducted when the subject visually recognizes the mark.

8. The optical measuring apparatus according to claim 4, further comprising:
a display that displays a mark at such a position that the eyeball is adducted when the subject visually recognizes the mark.

9. The optical measuring apparatus according to claim 1, further comprising:
an eyelid opening device that is brought into contact with a portion of skin near the eyeball of the subject so as to keep eyelids of the subject open.

10. The optical measuring apparatus according to claim 2, further comprising:
an eyelid opening device that is brought into contact with a portion of skin near the eyeball of the subject so as to keep eyelids of the subject open.

11. The optical measuring apparatus according to claim 3, further comprising:
an eyelid opening device that is brought into contact with a portion of skin near the eyeball of the subject so as to keep eyelids of the subject open.

12. The optical measuring apparatus according to claim 4, further comprising:
an eyelid opening device that is brought into contact with a portion of skin near the eyeball of the subject so as to keep eyelids of the subject open.

13. The optical measuring apparatus according to claim 5, further comprising:
an eyelid opening device that is brought into contact with a portion of skin near the eyeball of the subject so as to keep eyelids of the subject open.

14. The optical measuring apparatus according to claim 6, further comprising:
an eyelid opening device that is brought into contact with a portion of skin near the eyeball of the subject so as to keep eyelids of the subject open.

15. The optical measuring apparatus according to claim 7, further comprising:
an eyelid opening device that is brought into contact with a portion of skin near the eyeball of the subject so as to keep eyelids of the subject open.

16. The optical measuring apparatus according to claim 8, further comprising:
an eyelid opening device that is brought into contact with a portion of skin near the eyeball of the subject so as to keep eyelids of the subject open.

17. A method of outputting light and receiving the light, the method comprising:
outputting the light so as to cross an anterior chamber of an eyeball when the eyeball of a subject is adducted; and
receiving the light having crossed the anterior chamber.

18. The method according to claim 17, further comprising:
pressing a portion of skin near an inner canthus of the eyeball into an orbit that contains the eyeball,
wherein the light is output from a position reached by the portion of skin near the inner canthus having been pressed so as to cross the anterior chamber of the eyeball reaches.
